SEQUENCE LISTING (2 sequences)

SEQ ID NO: 1 - DNA, 3039 bp, Homo sapiens
  CDS (341)..(1144)
aataaagcgtgaacccgtccgtccggctcgcactttaagacttcccgagcggcggcggggacgccagtcgagccgggagacgcttacctgccgcttccccgcgccgcccggtgcacctggccgcaagggacctcgttctcagggaagacggcgacattccgcggaggtggaaccgccgcgcgccgtccgggctcggaccttccccggaacgtgggggcgccttagcgactccttccctgttgtgcccccgttcccggcgttcagcccggccccgcaaaggtgggacggctcccggcttcagttacggaagcggcccgtgtccagcgacgagggttcgaaaatgccccgcgcgttcctggtgaagaagccgtgcgtctccacgtgcaagaggaactggagcgagctccccgacgaggagcgcggcgagatctacgtgccagtcagcctgggcttctgcccaccacagccctaccgggagccggaaccctctgtggccgaacccccttcctgcccgctggctttgaacatgagccttcgagactctagctacagcatggcccccgggccctgtgtggtggcccagctgccctctgaagacatgggccacttgacagacccccagagcagagaccatggcttcctgcgcaccaagatgaaggtgacccttggggacagtcccagtggagacctgttcacctgccgtgtctgccagaaggccttcacctaccagcgcatgctgaaccgccacatgaagtgtcacaacgacgtcaagaggcacctctgcacgtactgcgggaagggcttcaatgacaccttcgacctcaagagacacgtccgaactcacactggcgtgcggccctacaagtgcagcctgtgtgacaaggccttcacgcagcgctgctctctggagtctcacctcaagaagatccatggtgtgcagcagaagtacgcgtacaaggagcggcgggccaagctgtacgtgtgtgaggagtgcggctgcacatctgagagccaggagggccacgtcctgcacctgaaggagcaccaccctgacagcccgctgctgcgcaagacctccaagaaggtggccgtggcactacagaacactgtcacttccctgctgcagggcagcccccacctgtgagtggctcgagccctgggggtgctcctggaagccccaagagcatccaggattgcctcccagctgcctggccagcccaccctcctgcaacctctcacccgaacaccagtgatcaggactggagcccccgtgccttggtctcccccctgggcacacgtgctcactcaggcccagcaatgacctctgctcatttttgcatttttgacttatgggccgaggctgttctgagcctgggaagatgtacctatgtcaagagaagggatgaggccaaggctgccttcaattagaagcagccgcccacagagacaggcactgtgtgcctggcagcaggacttcctacccagaggaggttcgagctaggatcccactgcccccgcctctcagcacagggcaggggctgcaggtccccagtggacatcagagtcaaaagcactggcaaagggtacccctgcaaacaactgtggtgggggctggcagcagaccccccacctggcagggcttctaatgctcagggttctggagggctctgtccttccggcaaggagaggcacacatgtgtgcccagccgtgtgtgtgcgtgtgcttgtgtgtgtgcactgctgtgtgtgtgtgcacgcacaggaagcctttccacatatcacctcatttctaagaaataaactacaaggtgccaagaaggttttatttccttttattttttaaagatgacaaatgtacagatgttaatatatttttggtgccaatggcgatgtttttaagagtgggatggagctggcttttctccattcccgtgcgcttctatttatcctggacatttcaaacctcctctgtgccttggctctgggcgggggctgccccaacccacccccgttctttgtacgtgctgagacagccactagaagatcttcctccagcggcgccctggacggctgctcctgcgaacagcccatggcatcttctgctcttccctcccggctctgccctgcacatcctgttgagccaagccccagtgacccggagagctggcctgatgctgagagtgtgtcctcctggggctttagggggcaggaaggtgggacgaatgacgatgcccatccactacctgaagcactaggacactcttgcagggccaggctggaagaccggcccttttcttggttgagtcaaaagccttagcacagtggcaaaaaatgggacagaatgatgaccagcacctcagaaacttccagagggaggagaggatttgatggctaccaaattgtatctgttgccttttttctgactttttcacctgaccaggctggggtttggagtggctgtggggagacccgtcctggctggctggctggctcccttgctcccttgctgcagctgggaaaggggttctgggtgtaaagaggtgtgcgtcttgtgggccaaagggaaaaacaggcaggggtcagagccagcctgccagaggcaaatgcaaaagaggtccccagaacacagccagctgggcagccccttaaagccaaaccccacctgaagcagaaccactttggcctcccctgcccaaaatgggtagtgtctacacgtccccgggctcaggctcaggcccagccctgggctgacctgagaggaaggctccttcctggactgccctctgaaatgtgtatagattgattctaaaatctcttgtttcacttgactttagagtgtctgggacgctgctgtattctgaaagtcacatagcacacagtaatgttatctggaagctctgtttttgtttacatttctgtatccctgggttgactgccaatccgaggccgtcatgaagctctgtgttgtctgttttattttataaccttcctctcaactattaaaattagagatctaatgtttaaaaaaaaaaaaaaaaa

SEQ ID NO: 2 - protein, 267 aa, Homo sapiens
MPRAFLVKKPCVSTCKRNWSELPDEERGEIYVPVSLGFCPPQPYREPEPSVAEPPSCPLALNMSLRDSSYSMAPGPCVVAQLPSEDMGHLTDPQSRDHGFLRTKMKVTLGDSPSGDLFTCRVCQKAFTYQRMLNRHMKCHNDVKRHLCTYCGKGFNDTFDLKRHVRTHTGVRPYKCSLCDKAFTQRCSLESHLKKIHGVQQKYAYKERRAKLYVCEECGCTSESQEGHVLHLKEHHPDSPLLRKTSKKVAVALQNTVTSLLQGSPHL

(19) United States
(12) Patent Application Publication
(10) Pub. No.: US 2016/0326587 A1
(43) Pub. Date: Nov. 10, 2016

(54) OVOL1 AS A NEW MARKER FOR MODERATE TO SEVERE ACNE

(71) Applicants: KING'S COLLEGE LONDON, London (GB); GALDERMA RESEARCH & DEVELOPMENT, Biot (FR)

(72) Inventors: JONATHAN BARKER, LONDON (GB); CATHERINE SMITH, LONDON (GB); RICHARD TREMBATH, LONDON (GB); ISABELLE CARLAVAN, BIOT (FR)

(21) Appl. No.: 15/105,031

(22) PCT Filed: Dec. 18, 2014
(86) PCT No.: PCT/EP2014/078422

§ 371 Date: Jun. 16, 2016

(30) Foreign Application Priority Data
Dec. 19, 2013 (EP) 13306779.3

(51) Int. Cl. C12Q 1/68

(52) U.S. Cl. CPC C12Q 1/6883; C12Q 2600/112; C12Q 2600/106; C12Q 2600/118; C12Q 2600/156; C12Q 2600/158

(57) ABSTRACT

The invention relates to the identification of the ovo-like zinc finger 1 (OVOL1) as a new biomarker of moderate to severe acne. The invention also relates to products and methods for detecting, diagnosing, staging, treating or monitoring the course of acne in a human subject.

The invention relates to the identification of the ovo-like zinc finger 1 (OVOL1) as a new biomarker of acne and to corresponding diagnostic and therapeutic applications as well as disease's management applications. The invention in particular relates to products and methods for detecting, diagnosing, staging or monitoring the course in vitro or ex vivo of acne in a subject, typically in a human subject. The invention further relates to products and methods for the determination of the presence or amount of OVOL1 in a biological sample. The invention also relates to binding reagents specific for OVOL1 and to compositions and devices containing the same. It further relates to the uses of these binding reagents, compositions and devices for acne detection, diagnostic, staging, monitoring, imaging or treatment, or for the determination of the presence or amount of OVOL1 in a biological sample, as well as for drug development.

The present invention more specifically relates to the assessment of acne using the DNA or the mRNA encoding OVOL1, or the OVOL1 protein, as a biomarker.

### BACKGROUND

Acne is a skin condition which results from the occlusion of the upper end and also of the internal part of the pilosebaceous canal owing to abnormal keratinocyte multiplication, and the androgenic hormone hyperactivity which often appears during puberty, which causes a considerable increase in seborrhoea in the sebaceous glands. The obstruction of the pilosebaceous canal causes the formation of comedones or microcysts, accompanied by proliferation of Propionibacterium acnes bacteria in the obstructed pilosebaceous follicles. This condition, which is particularly common in adolescents, is accompanied by an inflammatory reaction of the skin that may be in the form of papules or pustules generally located in the superficial dermis. In certain cases, the inflammatory reaction may reach the deep dermis, forming nodules and macrocysts.

Acne is the most common skin condition affecting millions of people worldwide. Patients with severe acne frequently face significant psychological and emotional problems due to the scarring associated with the disease. The pathogenesis of acne vulgaris is complex and incompletely understood.

The ovo-like zinc finger 1 (OVOL1) gene [NM004561 (mRNA) and NP-004552 (protein)] encodes C2H2 zinc finger transcription factor. Functional studies suggested that this gene plays important roles in the development of epithelial tissues and germ cells (Dai et al, 1998; Mevel-Ninio et al, 1995). Ovol1 acts downstream of the Wnt/β-catenin-signaling pathway that has been widely implicated in normal and malignant development of many tissues (Payre, et al, 1999). Additionally, OVOL1 was identified as a downstream target of the TGF-β/BMP7-Smad4 signaling pathway, a growth-inhibitory pathway in keratinocytes (Kowanetz et al, 2004). Ovol1 knockout mice display pleiotropic defects including ruffled hairs and a hyperproliferative epidermis (Nair et al, 2006).

In epidermis, Ovol1 is required for embryonic epidermal progenitor cells to efficiently exit proliferation to embark on the terminal differentiation process (Nair et al, 2006). Three downstream targets of Ovol1 have been identified: c-Myc, Id2 and Ovol2 (Li B et al, 2005; Nair et al, 2006). These genes are expressed in proliferating progenitor cells and their expression is up-regulated when Ovol1 is deleted. Both c-Myc and Id2 are known to have pro-proliferation and/or anti-differentiation roles, and therefore their negative regulation by Ovol1 is consistent with the growth inhibitory function of Ovol1.

Major contributors of acne pathogenesis are abnormal follicular differentiation with increased cornification, enhanced sebaceous gland activity with hyperseborrhea, bacterial hypercolonization, inflammation as well as immunological host reactions.

In latter events of inflammatory lesion development, the inflammatory response is down-regulated, allowing repair of the follicle through normal wound-healing mechanisms. In about ⅓ of individuals, healing of inflamed lesions leads to scarring (Cunliffe et al, 1998). The mechanisms behind this process remain unclear. The potential role of OVOL1 in acne pathogenesis is supported by the fact that OVOL1 is a downstream gene of TGFB signaling involved in keratinocyte proliferation and wound healing.

In the context of defect of healing mechanisms in acne scaring, there is a clear need for identifying new pharmacological markers allowing the correct and early diagnostic of acne as well as its adequate management, and new therapeutic targets allowing acne prevention, attenuation or treatment.

### BRIEF DESCRIPTION OF THE INVENTION

For the first time, inventors describe with experimental evidences OVOL1 as a biomarker (also herein identified as “marker”) for diagnosing moderate to severe acne, typically acne vulgaris. They further describe the modulation of its expression for treating acne.

A first object of the invention relates to the use of the DNA or the mRNA encoding OVOL1, or of the corresponding protein, as a biomarker for acne, typically for acne vulgaris. A second object of the invention relates to a method for the in vitro or ex vivo detection, diagnosis or staging of acne in an individual suspected of suffering of acne, comprising analysing the expression of OVOL1 in a biological sample from the individual, said analysis providing information on the presence or stage of acne in the individual.

Another object of the invention relates to a method for the in vitro or ex vivo detection or diagnosis of acne, comprising the steps of a) analysing the expression of OVOL1 in a biological sample from an individual suspected of suffering of acne; b) analysing the expression of OVOL1 in a biological sample from a healthy individual; and c) comparing the expressions of OVOL1 as analysed in steps a) and b); an expression of OVOL1 in the biological sample from the individual suspected of suffering of acne inferior to the expression of OVOL1 in the biological sample from the healthy individual being an indicator of the presence of acne in the individual suspected of suffering of acne, thereby detecting or diagnosing acne.

A further object of the invention relates to a method for monitoring in vitro or ex vivo the course of acne in an individual, wherein the method comprises a step of comparing the expression of OVOL1 in a first biological sample taken from an individual at t0 (initial time) to the expression of OVOL1 in a second biological sample taken from said individual at t1 (measure time after a while which can be expressed in minutes, hours, days, weeks or months), a decrease of the expression of OVOL1 in the sample taken at t1 being an indicator of the progression of acne in said individual and an increase of the expression of OVOL1 in the sample taken at t1 being an indicator of the regression of acne in said individual.

Another object of the invention relates to a method for monitoring in vitro or ex vivo the efficacy of a drug or composition for treating acne, comprising comparing the expression of OVOL1 in a first biological sample from an individual identified as having one or more of the symptoms of acne before any treatment of acne to the expression of OVOL1 in a second biological sample of the same individual who has been exposed to a drug or composition for treating acne, an increase in the expression of OVOL1 in the second biological sample being an indicator of efficacy of the drug or composition for treating acne, and a decrease in the expression or an absence of modulation of the expression of OVOL1 in the second biological sample being an indicator of inefficacy of the drug or composition for treating acne.

Also herein described is an in vitro or ex vivo screening method of a OVOL1 activator, comprising determining the ability of a drug candidate to activate or stimulate OVOL1 expression and/or OVOL1 biological function and if the ability is confirmed the identification of the drug candidate as a OVOL1 activator.

Another in vitro or ex vivo screening method of OVOL1 activators, comprises the following steps of: a) contacting a biological sample exhibiting an acne lesion, a biological sample exhibiting the healthy condition, or a mixture of said samples, with one or more drug candidates to be tested; b) detecting the expression and/or biological function of OVOL1 in the biological samples or mixture of samples of step a) and comparing said expression or biological function with the expression or biological function of OVOL1 in a sample which has not been contacted with the one or more drug candidates; and c) selecting as OVOL1 activators drug candidates which activate or stimulate the expression and/or biological function of OVOL1 as measured in the samples or mixtures obtained in the end of step a). The invention also relates to kits or devices suitable for implementing the above methods.

A further object of the invention relates to the use of an activator of OVOL1 for the preparation of a composition for treating acne.

Also herein described is an activator of OVOL1 for use for treating acne, typically acne vulgaris.

### FIGURES

FIG. 1 represents the regional association plots of Chr. 11q13.

FIG. 2 shows expression of OVOL1 in acne.

### DETAILED DESCRIPTION OF THE INVENTION

Inventors herein describe a new biomarker, the OVO-Like zinc finger 1 (OVOL1), which allows specific, reliable and sensitive detection and staging of acne in a subject, in particular in a human subject (also herein identified as human individual or human patient).

The OVOL1 protein is expressed in the epithelial tissues of hair follicles, interfollicular epidermis, kidney, as well as in the male germinal epithelium (Dai et al, 1998).

For the purpose of the present invention, the term “marker” or “biological marker” designates a biological marker associated with the presence or with the absence, presence or stage of a particular pathological state. A typical biological marker is in particular a protein, a mRNA or a DNA.

Unless otherwise specified, OVOL1 designates the OVOL1 gene, the OVOL1 mRNA or the OVOL1 protein as well as any fragment thereof. Specific examples of OVOL1 protein according to the invention include full length OVOL1 protein, and any fragment of interest thereof. The terms “OVOL1 expression” or “OVOL1 level of expression” refers to the presence, absence or amount of OVOL1 and means the level of mRNAs or proteins encoded by the gene marker. Such an expression is to be compared to a reference amount.

The inventors have herein analyzed the expression of OVOL1 in lesional skin of patients suffering from acne and compared said expression to that of non-lesional skin of the same patients. They surprisingly show a significant lower expression of OVOL1 transcripts in inflammatory papules of acne compared to normal skin of the same patients.

Altogether, the invention thus provides a novel biomarker, typically a novel acne biomarker, for clinical applications, in particular acne patient detection, diagnosis, monitoring and management.

Within the context of the invention, the use of OVOL1 for acne detection, diagnosis, staging or management (typically monitoring of the course of acne) includes, without limitation, the use of the protein (in any form, soluble or not, full length or not), or of any coding nucleic acids, as a biomarker. This includes, e.g., the use of any reagent to detect or quantify (i) the protein or any variant or mutant thereof, such as splicing variants or polymorphisms, and/or (ii) any nucleic acid encoding said proteins, such as DNA or RNA, said protein and/or nucleic acid levels being indicative of the absence of acne. The term also includes any measure of the expression level of the cited protein, and a comparison of the measured level to a reference or mean value. The measured amount or level or information provides an indication regarding acne in the subject.

The term “acne” herein typically refers to acne vulgaris (or simple acne) and/or to an acne associated disorder (e.g. hyperseborrhoea). The term “acne” also designates comedonic acne, papulopustular acne, papulocomedonic acne, nodulocystic acne, acne conglobata, cheloid acne of the nape of the neck, recurrent miliary acne, necrotic acne, neonatal acne, occupational acne, acne rosacea, senile acne, solar acne and medication-related acne.

A specific object of the invention relates to a method for the in vitro or ex vivo detection, diagnosis or staging of acne in an individual suspected of suffering of acne, comprising analysing the expression of OVOL1 in a biological sample from the individual, said analysis providing information on the presence or stage of acne in the individual.

Another object of the invention relates to a method for the in vitro or ex vivo detection or diagnosis of acne, comprising the following steps of:

a) analysing the expression of OVOL1 in a biological sample from an individual suspected of suffering of acne,

b) analysing the expression of OVOL1 in a biological sample from a healthy individual,

c) comparing the expressions of OVOL1 as analysed in steps a) and b), an expression of OVOL1 in the biological sample from the individual suspected of suffering of acne inferior to the expression of OVOL1 in the biological sample from the healthy individual being an indicator of the presence of acne in the individual suspected of suffering of acne, thereby detecting or diagnosing acne.

In the context of the invention, the individual is an animal, typically a mammal, preferably a human being, typically a patient, whatever its age or sex.

In the context of the invention, the biological sample corresponds to any type of sample taken from an individual, and can be a tissue sample or a fluid sample, such as blood, lymph or interstitial fluid. The biological sample is typically a tissue sample, such as a biopsy, in particular a skin biopsy, taken from an individual. The biopsy may vary in size and is preferably from 1 to 6 mm in diameter.

According to one particular and preferred embodiment, the sample is a skin sample taken by means of tape stripping, such as with D-Squames, according to the method described in Wong et al., 2006); or in Benson et al., 2006; or else in Wong et al., 2004. According to the principle of tape stripping, the product used comprises a flexible translucent polymer support and an adhesive. The product is applied repeatedly to the skin of the patient, preferably until loss of adhesion. The sample obtained relates only to the content of the outermost layers of the epidermis.

The analysis typically comprises determining the presence, absence or amount of OVOL1, the absence of OVOL1 or an expression thereof below a reference amount being indicative of the presence or stage of acne in the individual. In a particular embodiment, the presence of OVOL1 or an expression thereof above a reference amount is indicative of the absence of acne in the individual.

The OVOL1 expression analysis or detection can be performed by any suitable method, known to those skilled in the art, such as western blotting, IHC, mass spectrometry (Maldi-TOF and LC/MS analyses), radioimmunoassay (RIA), ELISA or any other method known to those skilled in the art or else by assaying the mRNA according to the methods customarily known to those skilled in the art. The techniques based on the hybridization of mRNA with specific nucleotide probes are the most customary [in situ hybridation, FISH, Northern blotting, RT-PCR (Reverse Transcriptase Polymerase Chain Reaction), quantitative RT-PCR (qRT-PCR), RNase protection.

The sequence analysis can be performed by any suitable method, known to those skilled in the art, such as next-generation sequencing (NGS).

A method for analysing a protein content obtained in particular according to the previously herein described sampling method is described in Patent Application WO2009/068825 (Galderma R&D). Since this method is rapid, non-invasive and relatively inexpensive, it is particularly preferred. Quantification is performed in the skin sample obtained on the flexible and adhesive support in order to detect the OVOL1 protein of which the presence, the absence or the variation in amount or in concentration compared with a standard value is associated with the presence, with the progression or with the absence of the (potentially suspected) acne.

In a particular embodiment, analysing OVOL1 comprises contacting a sample, or an aliquot thereof, with a specific binding reagent that binds a OVOL1 nucleic acid or protein and determining the presence or amount of OVOL1 nucleic acid or protein bound to said binding reagent.

Selective or specific binding indicates that binding to another molecule can be discriminated from (e.g., occurs with higher affinity or avidity than) specific binding to the target biomarker. Preferred reagents do not bind, under selective condition, to any other unrelated human protein but the reference protein. Binding of a reagent to a reference molecule can be tested according to methods well known by the skilled person.

The binding reagent is typically a specific ligand selected from a complementary nucleic acid, an antibody, an aptamer and a fragment or derivative thereof.

In a particular embodiment, the binding reagent is an antibody. The antibody may be a polyclonal or a monoclonal antibody, most preferably a monoclonal. It may be of various classes (e.g., IgG, IgE, IgM, etc.). The antibody may be of various animal origin, or human or synthetic or recombinant. Furthermore, the term antibody also includes fragments and derivatives thereof, in particular fragments and derivatives of said monoclonal or polyclonal antibodies having substantially the same antigenic specificity. Antibody fragments include e.g., Fab, Fab′2, CDRs, etc. Derivatives include humanized antibodies, human antibodies, chimeric antibodies, poly-functional antibodies, Single Chain antibodies (ScFv), etc. These may be produced according to conventional methods, including immunization of an animal and collection of serum (polyclonal) or spleen cells (to produce hybridomas by fusion with appropriate cell lines).

Methods of producing polyclonal antibodies from various species, including mice, rodents, primates, horses, pigs, rabbits, poultry, etc. are well known from the skilled person. Briefly, the antigen is combined with an adjuvant (e.g., Freud's adjuvant) and administered to an animal, typically by sub-cutaneous injection. Repeated injections may be performed. Blood samples are collected and immunoglobulins or serum are separated.

Methods of producing monoclonal antibodies from various species as listed above may be found, for instance, in Harlow et al., 1988 or in Kohler et al. 1975, incorporated herein by reference. Briefly, these methods comprise immunizing an animal with the antigen, followed by a recovery of spleen cells which are then fused with immortalized cells, such as myeloma cells. The resulting hybridomas produce the monoclonal antibodies and can be selected by limit dilutions to isolate individual clones. Antibodies may also be produced by selection of combinatorial libraries of immunoglobulins, as disclosed for instance in Ward et al. (Nature 341 (1989) 544).

Recombinant antibodies, or fragments or derivatives thereof, may be produced by methods known per se in the art, for example by recombination in a host cell, transformed with one or more vectors enabling the expression and/or secretion of the nucleotide sequences encoding the heavy chain or the light chain of the antibody. The vector generally contains a promoter, translation initiation and termination signals, and suitable transcriptional regulatory regions. It is stably maintained in the host cell and may optionally possess specific signals for secretion of the translated protein. These different components are selected and optimized by one of skill in the art according to the host cell used.

In a preferred embodiment, the anti-OVOL1 antibody, fragment or derivative thereof is an antibody, fragment or derivative thereof which binds human OVOL1. Specific examples of such antibodies include monoclonal antibodies.

Other antibodies may be found or generated against a OVOL1 protein and used in the present invention. It should be noted however that the use of antibodies that bind an epitope present in OVOL1 and wherein said binding is at least partially displaced by a human OVOL1 protein is particularly preferred as well as a fragment or derivative of such an antibody having the same antigen specificity.

For use in the invention, the antibodies may be coupled to heterologous moieties, such as labels, tags, linkers, etc., typically to a detectable moiety.

In a preferred embodiment, the complementary nucleic acid, fragment or derivative thereof binds all or part of SEQ ID NO:1 (human OVOL1 cDNA corresponding to OVOL1 mRNA), and the antibody, fragment or derivative thereof binds all or part of SEQ ID NO:2 (human OVOL1 protein).

The invention also relates to kits or devices suitable for implementing the above methods. A typical device comprises at least one specific reagent, typically at least one complementary nucleic acid, antibody, fragment or derivative thereof, that binds a OVOL1 nucleic acid or protein, said specific reagent being immobilized on a support. Preferably the support is a membrane, a slide, a microarray, a chip or a microbead.

A particular kit comprises a device as herein described and at least one reagent to perform, detect or quantify an immune reaction, in particular an antibody-antigen complex.

A further object of the invention relates to a method for monitoring in vitro or ex vivo the course of acne in an individual, wherein the method comprises a step of comparing the expression of OVOL1 in a first biological sample taken from an individual at t0 to the expression of OVOL1 in a second biological sample taken from said individual at t1, a decrease of the expression of OVOL1 in the sample taken at t1 being an indicator of the progression of acne in said individual and an increase of the expression of OVOL1 in the sample taken at tl being an indicator of the regression of acne in said individual.

Another object of the invention relates to a method for monitoring in vitro or ex vivo the efficacy of a drug or composition for treating acne, comprising comparing the expression of OVOL1 in a first biological sample from an individual identified as having one or more of the symptoms of acne before any treatment of acne to the expression of OVOL1 in a second biological sample of the same individual who has been exposed to a drug or composition for treating acne, an increase in the expression of OVOL1 in the second biological sample being an indicator of efficacy of the drug or composition for treating acne, and a decrease in the expression or an absence of modulation of the expression of OVOL1 in the second biological sample being an indicator of inefficacy of the drug or composition for treating acne.

Also herein described is an in vitro or ex vivo screening method of a OVOL1 activator, comprising determining the ability of a drug candidate to activate or stimulate OVOL1 expression and/or OVOL1 biological function and if the ability is confirmed the identification of the drug candidate as a OVOL1 activator.

Another in vitro or ex vivo screening method of OVOL1 activators, comprising the following steps of:

a) contacting at least one biological sample exhibiting an acne lesion, at least one biological sample exhibiting the healthy condition, or a mixture of said samples, with one or more drug candidates to be tested;

b) detecting the expression and/or biological function of OVOL1 in the biological samples or mixture of samples of step a) and comparing said expression or biological function with the expression or biological function of OVOL1 in a sample which has not been contacted with the one or more drug candidates; and

c) selecting as OVOL1 activators drug candidates which activate or stimulate the expression and/or biological function of OVOL1 as measured in the samples or mixtures obtained in the end of step a).

In a particular embodiment, two biological samples are collected and used in step a), one of said sample mimics acne lesion and the second one mimics the healthy condition.

The identified activator will influence the biological function of OVOL1 or a biological process activated by this biomarker. For screening purposes, the biological samples advantageously consist of transfected cells containing reporter genes operating under the control of a promoter (totally or partially) controlling the expression of the OVOL1 gene. Alternatively, the promoter may be, at least in part, synthetically assembled and contain OVOL1-responsive elements. The ability of a compound to modulate the function of OVOL1 is evaluated by analysing the expression of the reporter gene.

The transfected cells may further be engineered to express the OVOL1 protein.

The reporter gene may encode an enzyme that with its corresponding substrate, provides coloured product(s) such as CAT (chloramphenicol acetyltransferase), GAL (beta galactosidase), or GUS (beta glucuronidase). It might be either luciferase or GFP (Green Fluorescent Protein). Reporter gene protein dosage or its activity is typically assessed by colourimetric, fluorometric or chemoluminescence methods.

A further object of the invention relates to the use of an activator of OVOL1, typically identified by screening methods as defined above, for the preparation of a composition for treating acne. Also herein described is an activator of OVOL1 for use for treating acne, typically acne vulgaris.

The selected activator or agonist can be a polypeptide, a DNA, an RNA, or a PNA (“Peptide nucleic acid”, i.e. a DNA-like structure with a polypeptidic chain substituted by purine and pyrimidine bases). Advantageously, the activator or agonist is administered to a patient in a sufficient quantity so as the measure a plasmatic concentration. This quantity will be easily determined by the skilled person depending on the subject.

The invention relates to the use of the DNA or the mRNA encoding OVOL1, or the corresponding protein, as markers, more particularly as biomarkers for acne, typically acne vulgaris.

The invention also provides a method for diagnosing acne and/or assessing the severity of acne in a patient comprising the step of assessing: (a) the level of expression; (b) the activity; or (c) the sequence of OVOL1 gene, promoter and/or expression product.

For example, the method for diagnosing acne, may comprise the following steps:

a) analysing the level of expression or the activity or the sequence of OVOL1 including exons, introns, upstream and downstream non-coding regions involved in the regulation of OVOL1 expression, in a biological sample from an individual,

b) analysing the level of expression or the activity or the sequence exons, introns, upstream and downstream non-coding regions involved in the regulation of OVOL1 expression in the regulation of OVOL1 expression, in a biological sample from a healthy individual,

c) comparing the difference in level of expression or activity or in sequence of OVOL1 including exons, introns, upstream and downstream non-coding regions involved in the regulation of OVOL1 expression to healthy individual,

d) the difference of step c) is indicative of acne disease or a predisposition to acne, thus diagnosing acne vulgaris.

The invention provides a method for monitoring the progression of acne, comprising the following steps:

a) taking a biological sample from the individual,

b) analysing level of expression or the activity or the sequence of OVOL1 including exons, introns, upstream and downstream non-coding regions involved in the regulation of OVOL1 expression in a sample taken and in which a variation in the expression or activity or in the sequence of OVOL1 including introns, upstream and downstream non-coding regions involved in the regulation of OVOL1 expression is an indicator of the progression of acne.

Progression of acne may be from a predominantly comedonal to a more inflammatory dominated state, it may also mean progression towards specific acne subtypes, like nodulocystic acne or acne conglobata for example. Progression might also occur in the other direction, from a more severe to a less severe form of acne.

The invention provides also a method for monitoring the efficacy of a treatment intended for treating acne, comprising the following steps:

a) administering the desired treatment to the individual identified as having one or more of the symptoms of acne,

b) taking a biological sample from the individual,

c) analysing the level of expression or the activity or the sequence of OVOL1 including exons, introns, upstream and downstream non-coding regions involved in the regulation of OVOL1 expression, in which a variation in the expression, activity or sequence of this marker is an indicator in the treatment of acne.

Another embodiment of the present invention is in vitro screening method of modulators of OVOL1 for treating acne, comprising determining the capacity of said candidate to activate or up-regulate expression or to improve biological activity of OVOL1. That includes activators of signalling pathways leading to increase in OVOL1 transcription, such as TGFb signalling, BMP signalling.

More specifically, the invention provides an in vitro screening method of OVOL1 activators for the identification of drug candidates, comprising the following steps:

a) Collecting at least one biological sample;

b) Contacting at least one sample or a mixture of samples with one or more drug candidates to be tested;

c) Detecting the expression or biological function of OVOL1 in the biological samples or mixture obtained in b);

d) Selecting drug candidates, which are capable of increasing the expression or activating biological function of OVOL1 measured in said samples or mixtures obtained in b) and comparing the levels with a sample not mixed with the drug candidate (s).

For the screening, biological samples are transfected cells containing reporter gene operably under the control of a promoter (totally or partially) controlling the expression of OVOL1 gene. Therefore step c) above consists in measuring the OVOL1 expression of the reporter gene.

The reporter gene may encode an enzyme that with its corresponding substrate, provides coloured product(s) such as CAT (chloramphenicol acetyltransferase), GAL (beta galactosidase), or GUS (beta glucuronidase). It might be either luciferase or GFP (Green Fluorescent Protein) gene.

Reporter gene protein dosage or its activity is typically assessed by colouring, fluorometric or chemoluminescence methods.

Biological samples are also cells expressing the gene of interest and the step c) above consists to measure the activity of the gene product.

Any kind of cell is suitable for the invention. Cells may endogenously express the said gene like keratinocytes. Organs may be suitable for the instant invention, from animal or human origin like skin.

Transformed cells by heterologous nucleic acid encoding the gene expression product of interest might be suitable. Preferably the said nucleic acid is from animal (preferred mammal) or human origin. A large variety of host cells is suitable for the invention and in particular Cos-7, CHO, BHK, 3T3, HEK293 cells. Cells may be transiently or permanently transfected by a nucleic acid of interest with a well-known by those skilled in the art method and for instance calcium phosphate precipitation, DEAE-dextran, liposome, virus, electroporation or microinjection.

The gene expression of step c) is determined with the same techniques quoted above.

The compounds to be tested are any kind of compounds, from natural or synthetic source. As synthetic compounds they might be chemically synthesized or from a chemical compound data bank, with a defined structure or non-characterized or present in a mixture of compounds. Molecules improving the expression of OVOL1 or its biological activity or biological function can also be provided by all systems and methods modulating the expression of OVOL1 such as methods of gene replacement, gene therapy and delivery of therapeutic protein, comprising OVOL1 (miRNA, mRNA, DNA, protein).

Another embodiment of the present invention is an in vitro screening method of OVOL1 activators, comprising determining the capacity of said candidate to activate and/or up-regulate the expression or the biological activity or the biological function, including the transactivation properties, of the proposed marker of the invention.

According to a further embodiment of the invention, biological samples are cells expressing the gene of interest and the step c) above consists to measure the activity of the gene product.

In another particular embodiment, the invention relates to the use of identified activators with the described screening methods for the preparation of a composition for treating acne and/or acne associated disorders. Preferably the identified activator is a polynucleotide, a polypeptide, an antibody or a small organic molecule.

The present description also concerns a method for treating acne using an activator of OVOL1 as herein described.

The examples, which follow, illustrate the invention without limiting the scope thereof.

### EXAMPLES

### Example 1

### GWAS Study

Since, twin and family studies indicate that a family history doubles the risk of significant acne, we performed the first GWAS in moderate-to-severe acne in order to identify the predisposing genetic architecture.

Patients:

Patients had a diagnosis of acne vulgaris made by a trained dermatologist, with at least moderate severity as defined by the presence of nodulocystic disease and/or Leeds Grade >5 severity and/or requiring treatment with isotretinoin and or presence of severe forms of acne.

Samples: A total of 4,208 samples were assembled for this investigation, of which 2,001 samples from unrelated individuals of European ancestry were processed for the GWAS discovery set and 2,207 of which were processed for the second stage set. A total of 1,894 DNA samples, recruited from 17 centers from the UK were included in the GWAS discovery set, passed pre- and post-genotyping control filters (see below). A total of 2063 DNA samples were included in the second stage dataset. Phenotypic data and blood samples were collected after research ethics approval was received from each participating institution and after subjects had given written informed consent.

Controls: A total of 7,271 control individuals passed the quality control filters (see below). 5,139 individuals from the WTCCC2 common control set were used in the discovery GWAS. This included 2478 healthy blood donors from the United Kingdom Blood Service (UKBS) collection and 2661 individuals from the 1958 Birth Cohort (58C) dataset. 2,132 individuals enriched for no history of acne from the Twins UK registry were used in the second stage dataset.

Quality Control:

Samples. We used plink and principal component analysis with Eigensoft to detect and exclude outlying individuals on the basis of call rate, heterozygosity, relatedness, sex mismatches and ancestry. We also excluded one of each pair of related individuals.

SNPs. SNPs were excluded if the Fisher information for the allele frequency was not close to unity or for extreme departures from Hardy-Weinberg equilibrium (see below). Cluster plots of SNPs showing putative associations were inspected manually. The quality control measures excluded 251 cases and 169 controls. 31% (255193) non-overlapping SNPs were excluded during merging of case and control datasets, an additional 1.5% (8588) SNPs were excluded during QC in plink due to missingness thresholds of <95% and/or Hardy Weinberg

Equilibrium test with p<10-6.

Statistical methods. We performed principal component analysis on a subset of 83,484 post-quality-control SNPs (none from the MHC region), selected so as to minimize the contribution from regions of extensive strong linkage desequilibrium and to ensure that only genome-wide effects were detected. Principal component scores were computed for the combined dataset of post-exclusion case and control samples. After inspection, the first four principal components did not lead to differentiation of individuals by geographical origin, suggesting negligible differences in ancestry within the dataset. However, a part of the acne cases clustered outside of the bulk of the plot without showing geographical or ethnical differences to the majority of cases. To guard against possible artefacts, we repeated the primary association analysis excluding these cases. Reassuringly, this yielded comparable association signals. As we expected genotyping-platform dependent differences between cases and controls, we chose to use the first four principal components. However, no single principal component showed significant differences between cases and control.

We used a logistic regression model with case or control status as the response variable, the first four principal components as covariates and the genotype at a particular SNP as the explanatory variable. Genome-wide association tests were carried out at each SNP with uncertainty in genotype calls modelled using missing data likelihoods as implemented in SNPTEST. Unless otherwise stated, we assumed that the change in the odds of case status due to each copy of the allele was multiplicative.

It has become standard practice in GWAS to refer to the odds ratio associated with a particular allele or haplotype, which we estimate as eβ, where β is the maximum likelihood estimate of the coefficient describing the effect of each predictor on the response in the assumed model. We note however that, as is true of this study and many others, where the controls are taken at random from the population without reference to disease status, β is actually the log of the relative risk and not the log of the odds ratio.

Imputation was performed in a two-stage approach using phasing with Shapelt and imputation using IMPUTE2 on the 1000 Genomes reference panel. We used frequentist conditional analyses to look for primary and secondary association signals at known and putative SNPs. Selection of SNPs for replication was performed using the threshold of p value <10-4 in the discovery set, prioritizing genotyped over imputed SNPs, SNPs with a minor allele frequency of >0.02, as well as cross-referencing with lists of candidate genes that had arisen in transcription studies of acne biopsies and sebocyte cultures. We aimed to have at least two SNPs with r2>0.8 per region in case one should fail to be genotyped in the second stage cohort.

We used standard ‘fixed-effect’ meta-analysis techniques to analyse the second stage data. We fitted a logistic regression model for case or control status with no covariates at each SNP with a single parameter for the genetic effect (a multiplicative effect on the risk scale and an additive effect on the log-odds scale). As is typical for GWAS second stage studies which type a small number of SNPs, testing for possible substructure within populations was not possible.

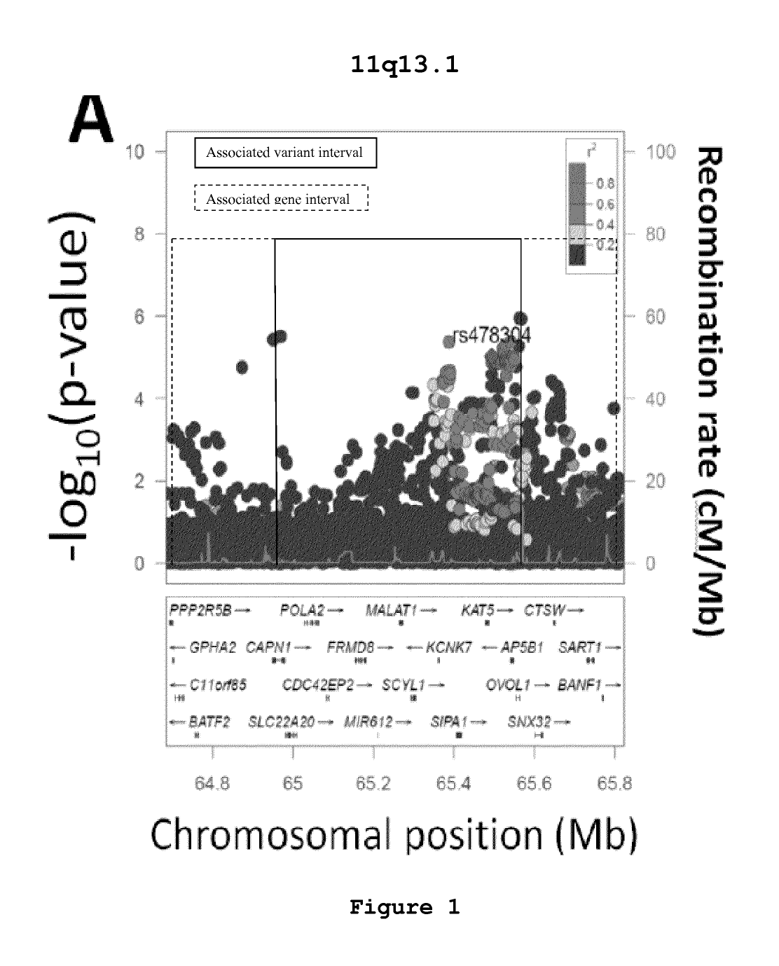

FIG. 1: This figure represents the regional association plots of Chr. 11q13. The −log 10 P values for the SNPs at the new locus are shown on the left y axis of each plot.

SNPs are coloured based on their r2 with the labelled hit SNP which has the smallest P value in the region. r2 is calculated from the 1000 Genomes (March 2012) genotypes. The bottom section of each plot shows the fine scale recombination rates estimated from individuals in the 1000 Genomes population, and genes are marked by horizontal blue lines

Table 1 provides locus showing Genome-wide significant association with acne.Genome-wide significant associations identify a genomic region, from 64.8 Mb to 65.8 Mb, on 11q13.1 (rs478304) with P combined=3.23×10-11. Among known genes encompassed in this region, OVOL1, which is very close to the lead SNP, is of major interest.

TABLE 1

| | | | Gene of | Risk | RAF | | Discovery sample | | Second stage sample | | Combined |
|---|---|---|---|---|---|---|---|---|---|---|---|
| Chr | rsID | Position | Interest | allele | Cases | Controls | P_{scan} | OR (95% CI) | P_{2nd stage} | OR (95% CI) | P_{com} |
| 11q13.1 | rs478304 | 65′494′260 | OVOL1, | T | 0.6 | 0.55 | 9.58 × 10⁻⁶ | 1.20 (1.11-1.29) | 2.65 × 10⁻⁷ | 1.26 | 3.23 × 10⁻¹¹ |
| | | | others | | | | | | | (1.16-1.38) | |

### Example 2

### Analysis of the Expression of OVOL1 in the Lesional Skin of Patients Suffering From Acne Compared With Non-Lesional Skin of These Patients

To gain a better understanding of acne pathogenesis, an Affymetrix study was performed on inflammatory papules and non lesional skin.

Patient Selection and Tissue Biopsies:

Skin biopsies of acne patients were obtained from an inflammatory papule and from non lesional skin in 12 patients with acne, in accordance with good clinical practice. (The clinical description of acne subtypes was carried out according to the classification of Wilkin et al., 2002.)

To evaluate a change in the expression level of the genes, the expression levels in lesional skin are compared with the expression levels in non-lesional skin of the same subjects (n=12).

mRNA Extraction, Labelling and Hybridization to Probe Arrays:

The mRNA was isolated from skin using the RNeasy extraction kit (Qiagen Inc., Valencia, Calif.) and quality was evaluated using a 2100 Bioanalyser of Agilent. The mRNA expression was evaluated by a Gene Chip IVT labelling kit after the generation of double-stranded cDNA (i.e. in vitro transcription process) using T7-oligo primer and the one cycle cDNA synthesis kit of Affymetrix. RNA was ethanol precipitated to concentrate the sample and then quantified using a spectrophotometer. Approximately 200 ng of total RNA of good quality [RNA indication number (RIN)≧7] from each sample was used to generate double-stranded cDNA using a T7-oligo (dt) primer (one cycle cDNA synthesis kit, Affymetrix). Biotinylated cRNA, produced through in vitro transcription (Gene Chip IVT labelling kit, Affymetrix) was fragmented and hybridised to an Affymetrix human U133A 2.0 plus microarray. The arrays were processed on a Gene Chip Fluidics Station 450 and scanned on an Affymetrix Gene Chip Scanner (Santa Clara, Calif.).

Statistical Analysis of mRNA Expression Based on Affymetrix Gene Chips:

The expression data from Affymetrix Gene Chips are normalized with RMA (Robust Multi-array Analysis) method. The raw intensity values are background corrected, log 2 transformed and then quantile normalized. Next a linear model is fit to the normalized data to obtain an expression measure for each probe set on each array. To identify genes that were significantly modulated in the different Acne subtype samples, one-way ANOVA with Benjamini-Hochberg multiplicity correction was performed using JMP 7.0.1 (SAS Institute) and irMF 3.5 (National Institute of Statistical Sciences, NISS) software.

Table 2: this table collects data of OVOL1 mRNA expression measured by Affymetrix in acne lesion and non-involved skin.

TABLE 2

| | | | Normalized Expression by RMA in Non-Lesional Skin | | | | | | | | |
|---|---|---|---|---|---|---|---|---|---|---|---|
| | | | | | | | | | | Patient | Patient |
| Probe Set | GENE_SYMBOL | TITLE | Patient 1 | Patient 2 | Patient 3 | Patient 4 | Patient 5 | Patient 6 | Patient 7 | 8 | 9 |
| 229396_at | OVOL1 | ovo-like 1 | 1151 | 977 | 1001 | 1076 | 1118 | 1527 | 1012 | 839 | 1324 |

| | | | Normalized Expression by | | | | | | | | |
|---|---|---|---|---|---|---|---|---|---|---|---|
| | | | RMA in Non-Lesional Skin | | | | | | | | |
| | | | | Patient | Patient | Normalized Expression by RMA in Lesional Skin | | | | | |
| Probe Set | GENE_SYMBOL | TITLE | Patient 10 | 11 | 12 | Patient 1 | Patient 2 | Patient 3 | Patient 4 | Patient 5 | Patient 6 |
| 229396_at | OVOL1 | ovo-like 1 | 1220 | 1309 | 1058 | 936 | 911 | 1032 | 485 | 1055 | 1464 |

| | | | | | | | | | | | Lesional | [symbol] |
|---|---|---|---|---|---|---|---|---|---|---|---|---|
| | | | | | | | | | Mean | | Skin | [symbol] |
| | | | | | | | | | expression | Mean | vs | [symbol] |
| | | | | | | | | | In | expression | Non- | [symbol] |
| | | | Normalized Expression by RMA in Lesional Skin | | | | | | Non- | In | Lesional | [symbol] |
| | | | Patient | Patient | Patient | Patient | Patient | Patient | Lesional | Lesional | Skin | [symbol] |
| Probe Set | GENE_SYMBOL | TITLE | 7 | 8 | 9 | 10 | 11 | 12 | Skin | Skin | Fold_Charge | [symbol] |
| 229396_at | OVOL1 | ovo-like 1 | 972 | 459 | 751 | 1617 | 869 | 766 | 1130 | 887 | −1.3 | [symbol] |

[symbol]

indicates data missing or illegible when filed

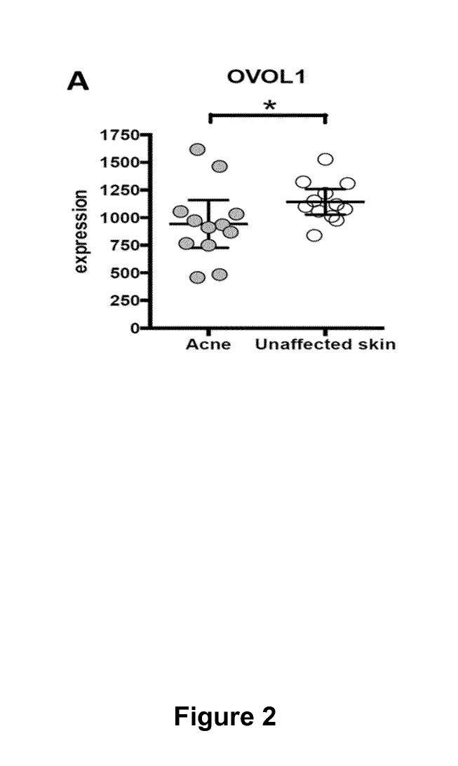

FIG. 2 presents the expression of OVOL1 in acne.

Normalized expression of probe set for OVOL1 on Affymetrix RNA expression chips (229396_at OVOL1,) in 12 biopsies of inflammatory acne papules compared with uninvolved skin of the same patients. Analysis with double paired t-test with Benjamini-Hochberg correction (*Pvalue<0.05).

Table 2 and FIG. 2 show statistically significant lower expression of OVOL1 transcripts in inflammatory papules of acne compared to normal uninvolved skin of the same patients.

Altogether, the GWAS results and the lower expression of OVOL1 in acne lesions support a role of OVOL1 in acne pathology.

### REFERENCES

Benson N R, et al., “An analysis of select pathogenic messages in lesional and non-lesional psoriatic skin using non-invasive tape harvesting”. J Invest Dermatol. 2006 October; 126(10): 2234-41.

Cunliffe W J. The sebaceous gland and acne—40 years on. Dermatology 1998; 196:9-15.

Dai, X., C. Schonbaum, L. Degenstein, W. Bai, A. Mahowald, and E. Fuchs. 1998. The ovo gene required for cuticle formation and oogenesis in flies is involved in hair formation and spermatogenesis in mice. Genes Dev. 12:3452-3463.

Harlow et al. “Antibodies: A laboratory Manual”, CSH Press, 1988.

Kohler et al. Nature 256 (1975) 495.

Kowanetz, M., U. Valcourt, R. Bergstrom, C. H. Heldin, and A. Moustakas. 2004. Id2 and Id3 define the potency of cell proliferation and differentiation responses to transforming growth factor beta and bone morphogenetic protein. Mol. Cell. Biol. 24:4241-4254.

Li B, Nair M, Mackay D R, Bilanchone V, Hu M, Fallahi M, Song H, Dai Q, Cohen P E, et al. Ovol1 regulates meiotic pachytene progression during spermatogenesis by repressing Id2 expression. Development. 2005; 132:1463-1473.

Mevel-Ninio, M., R. Terracol, C. Salles, A. Vincent, and F. Payre. 1995. Ovo, a Drosophila gene required for ovarian development, is specifically expressed in the germline and shares most of its coding sequences with shavenbaby, a gene involved in embryo patterning Mech. Dev. 49:83-95.

Nair M, Teng A, Bilanchone V, Agrawal A, Li B, Dai X. Ovol1 regulates the growth arrest of embryonic epidermal progenitor cells and represses c-myc transcription. J. Cell. Biol. 2006; 173:253-264.

Nair, M. et al. Ovol1 regulates the growth arrest of embryonic epidermal progenitor cells and represses c-myc transcription. J Cell Biol 173, 253-64 (2006).

Payre, F., A. Vincent, and S. Carreno. 1999. Ovo/svb integrates Wingless and DER pathways to control epidermis differentiation. Nature. 400:271-275.

Teng A, Nair M, Wells J, Segre J A, Dai X. Strain-dependent perinatal lethality of Ovol1-deficient mice and identification of Ovol2 as a downstream target of Ovol1 in skin epidermis. Biochim. Biophys. Acta. 2007; 1772:89-95).

Wilkin et al., 2002, J. Am. Acad. Dermatol. Vol 46, pages 584-587.

Wong R et al., “Analysis of RNA recovery and gene expression in the epidermis using non-invasive tape stripping”; J Dermatol Sci. 2006 November; 44(2):81-92.

Wong R et al., “Use of RT-PCR and DNA microarrays to characterize RNA recovered by non-invasive tape harvesting of normal and inflamed skin”. J Invest Dermatol. 2004 July; 123(1):159-67.

1-15 (canceled)

16. A method for the in vitro or ex vivo detection, diagnosis or staging of acne in an individual suspected of suffering of acne, comprising analysing the expression of OVOL1 in a biological sample from the individual, said analysis providing information on the presence or stage of acne in the individual.

17. The method of claim 16, wherein the analysis comprises determining the presence, absence or amount of OVOL1, the absence of OVOL1 or an expression thereof below a reference amount being indicative of the presence or stage of acne in the individual.

18. The method of claim 16, wherein analysing OVOL1 comprises contacting a sample, or an aliquot thereof, with a specific binding reagent that binds a OVOL1 nucleic acid or protein and determining the presence or amount of OVOL1 nucleic acid or protein bound to said binding reagent.

19. The method of claim 18, wherein the binding reagent is selected from a complementary nucleic acid, an antibody, and a fragment or derivative thereof.

20. The method of claim 19, wherein the complementary nucleic acid, fragment or derivative thereof binds all or part of SEQ ID NO:1 (human OVOL1 cDNA), and the antibody, fragment or derivative thereof binds all or part of SEQ ID NO:2 (human OVOL1 protein).

21. A device comprising at least one complementary nucleic acid, antibody, fragment or derivative thereof that binds a OVOL1 nucleic acid or protein immobilized on a support.

22. A kit comprising a device according to claim 21 and a reagent to perform, detect or quantify an immune reaction, or an antibody-antigen complex.

23. A method for the in vitro or ex vivo detection or diagnosis of acne, comprising the following steps of:
a) analysing the expression of OVOL1 in a biological sample from an individual suspected of suffering of acne,
b) analysing the expression of OVOL1 in a biological sample from a healthy individual, and
c) comparing the expressions of OVOL1 as analysed in steps a) and b), an expression of OVOL1 in the biological sample from the individual suspected of suffering of acne inferior to the expression of OVOL1 in the biological sample from the healthy individual being an indicator of the presence of acne in the individual suspected of suffering of acne, thereby detecting or diagnosing acne.

24. A method for monitoring in vitro or ex vivo the course of acne in an individual, wherein the method comprises a step of comparing the expression of OVOL1 in a first biological sample taken from an individual at t0 to the expression of OVOL1 in a second biological sample taken from said individual at t1, a decrease of the expression of OVOL1 in the sample taken at t1 being an indicator of the progression of acne in said individual and an increase of the expression of OVOL1 in the sample taken at t1 being an indicator of the regression of acne in said individual.

25. A method for monitoring in vitro or ex vivo the efficacy of a drug or composition for treating acne, comprising comparing the expression of OVOL1 in a first biological sample from an individual identified as having one or more of the symptoms of acne before any treatment of acne to the expression of OVOL1 in a second biological sample of the same individual who has been exposed to a drug or composition for treating acne, an increase in the expression of OVOL1 in the second biological sample being an indicator of efficacy of the drug or composition for treating acne, and a decrease in the expression or an absence of modulation of the expression of OVOL1 in the second biological sample being an indicator of inefficacy of the drug or composition for treating acne.

26. An in vitro or ex vivo screening method of a OVOL1 activator, comprising determining the ability of a drug candidate to activate or stimulate OVOL1 expression and/or OVOL1 biological function and if the ability is confirmed the identification of the drug candidate as a OVOL1 activator.

27. An in vitro or ex vivo screening method of OVOL1 activators, comprising the following steps of:
a) contacting a biological sample exhibiting an acne lesion, a biological sample exhibiting the healthy condition, or a mixture of said samples, with one or more drug candidates to be tested;
b) detecting the expression and/or biological function of OVOL1 in the biological samples or mixture of samples of step a) and comparing said expression or biological function with the expression or biological function of OVOL1 in a sample which has not been contacted with the one or more drug candidates; and
c) selecting as OVOL1 activators drug candidates which activate or stimulate the expression and/or biological function of OVOL1 as measured in the samples or mixtures obtained in the end of step a).

28. A method for treating acne in a subject comprising a step of administering an activator of OVOL1 to the subject.
